(19) United States
(12) Patent Application Publication
(10) Pub. No.: US 2004/0109824 A1
(43) Pub. Date: Jun. 10, 2004

(54) Particles for imaging cells

(76) Inventors: Kathleen Allison Hinds, Rockville, MD (US); Cynthia E. Dunbar, Washington, DC (US)

Correspondence Address: MERCHANT & GOULD PC, P.O. BOX 2903, MINNEAPOLIS, MN, 55402-0903, US

(21) Appl. No.: 10/313,304

(22) Filed: Dec. 6, 2002

(51) Int. Cl. A61K 49/00

(52) U.S. Cl. 424/9.32; 424/9.6

(57) ABSTRACT

The invention includes a particle that includes at least one magnetic resonance imaging (MRI) active material, at least one fluorescent material, and at least one polymer. The invention also includes a method of tracking at least one cell that includes labeling the cell with a particle in accordance with the invention, and monitoring the cell using magnetic resonance imaging (MRI), flow cytometry, fluorescent techniques, microscopy studies, or combinations thereof.

This invention is supported by the Department of Health and Human Services. The Government of the United States of America may have certain rights in the invention disclosed and claimed herein below.

### FIELD OF THE INVENTION

The invention relates to particles for imaging cells. More specifically, the invention relates to particles that can be used to image cells with a number of imaging techniques, such as, for example magnetic resonance imaging, flow cytometry, and microscopy studies.

### BACKGROUND OF THE INVENTION

Tracking of individual cells within a subject, such as a human or an animal may offer important information on a number of biological and/or clinically important processes. For example, in the case of bone marrow transplants, tracking of hematopoietic stem and/or progenitor cells in vivo could offer important insight into this biologically complex and clinically important procedure.

Tracking of cells in vivo can be accomplished with a number of different techniques. Once the cell has reached its final destination, magnetic resonance imaging (“MRI”) can be used to monitor the cells in their microenvironment. To obtain a more clear picture of the fate of the cells in vivo it would be helpful to be able to utilize other techniques as well. For example, the use of flow cytometry would allow the monitoring of intracellular distribution and sorting of the cells based on whether or not they had been labeled. Furthermore, the use of fluorescence or confocal microscopy could be utilized to further locate and image cells.

Progress has recently been made in labeling cells with substances containing iron oxide that can be imaged by MRI. However, an optimal method has not yet been found. Most commonly used MRI cell tracking studies label cells with ultrasmall (nanometer diameters) dextran coated iron oxide particles that enter cells through endocytosis. The resolution and sensitivity of MRI to detect small numbers of cells or individual cells is dependent on both the size of the particles and the efficiency of their uptake by cells. Therefore, for a given amount of iron, the ultrasmall particles such as the dextran coated particles, have been disappointing for imaging small numbers of cells using MRI.

An example of some experiments that utilize these ultra small particles for MRI imaging include the following. Seneterre et al. utilized ultrasmall superparamagnetic iron oxide particles for MR imaging both in vivo and in vitro. Senéterre et al, Bone Marrow: Ultrasmall superparamagnetic iron oxide for MR Imaging, Radiology 1991; (179) p. 529. Similarly, Dodd et al. utilized dextran-coated superparamagnetic particles to detect single mammalian cells. Dodd et al., Detection of Single Mammalian Cells by High-Resolution Magnetic Resonance Imaging, Biophysical Jrnl. 1999; (76) p. 103. Lewin et al. tracked and recovered progenitor cells by using small (5 nm) particles with a monocrystalline superparamagnetic iron oxide core coated with a crosslinked aminated dextran. Lewin et al., Tat peptide-derivatized magnetic nanoparticles allow in vivo tracking and recovery of progenitor cells, Nature Biotechnology 2000 (18) p. 410.

A number of superparamagnetic particles have also been the subject of issued patents, such as those discussed below. Yudelson (U.S. Pat. No. 4,965,007) discloses a superparamagnetic magnetite particles having diameters between 0.005 μm and 0.035 μm. Unger (U.S. Pat. Nos. 5,358,702 and 5,976,500) teaches a gel particle with diameters less than 90 μm that include a polymer that entraps at least one contrast enhancing metal. Similarly, Lauterbur et al. (U.S. Pat. No. 5,532,006) discloses a diagnostic agent with a matrix and a magnetic component such as magnetite.

The previously discussed particles are generally small, and therefore produce a relatively small MRI signal. Furthermore, none of the particles above include components that can be used to monitor the cell using other techniques, such as flow cytometry, fluorescence microscopy or confocal microscopy.

Therefore, there remains a need for a particle, that can be used to track a cell in vivo or in vitro, that exhibits an acceptable MRI signal and can also be tracked with other methods such as flow cytometry, fluorescence microscopy, or confocal microscopy.

### SUMMARY OF THE INVENTION

The invention provides a particle that includes at least one magnetic resonance imaging (MRI) active material, at least one fluorescent material, and at least one polymer.

The invention also provides methods of tracking at least one cell in an environment that includes the steps of labeling the cell with a particle that includes at least one MRI active material, at least one fluorescent material, and at least one polymer, and monitoring the labeled cell within the environment by monitoring the particle using magnetic resonance imaging (MRI), flow cytometry, fluorescent techniques, and microscopy studies.

The particles of the invention can be used to label and track cells. These particles are generally more effective for MRI imaging than those previously used because they are more effective as a contrast agent. Their increased effectiveness as a contrast agent results from their larger size relative to prior art particles previously utilized. The particles of the invention also include a fluorescent material. The inclusion of a fluorescent material allows tracking by flow cytometry, fluorescent microscopy, and confocal microscopy as well as MRI. Because the particle allows multiple methods of tracking to be utilized, the cells can be tracked on multiple spatial scales from intracellular distribution to the distribution within the subject, as well as in cell sorting devices for the selection of appropriately labeled cells.

### BRIEF DESCRIPTION OF THE DRAWINGS

FIG. 1 depicts signal intensities of T₂* weighted images of dilutions of Tendex™ particles and particles in accordance with the invention.

FIG. 2A shows light microscopy images of CD34+ cells labeled with a particle in accordance with the invention.

FIG. 2B shows a fluorescent micrograph of CD34+ cells labeled with a particle in accordance with the invention.

FIG. 2C shows a higher power fluorescent micrograph of the same field as FIG. 2B.

FIG. 3A is a confocal fluorescence micrograph of CD34+ cells labeled with a particle in accordance with the invention.

FIG. 3B is an electron micrograph of a single CD34+ cell labeled with a particle in accordance with the invention.

FIG. 4A is a green fluorescence image of mesenchymal stem cells labeled with particles in accordance with the invention.

FIG. 4B is a red fluorescence image of MSCs labeled with endosomal marker CM-DiI.

FIG. 4C is a fluorescence image showing both CM-DiI and particles in accordance with the invention in MSCs.

FIG. 4D is a Nomanski optics view of MSCs labeled with particles in accordance with the invention.

FIGS. 5A and B depict growth of CD34+ cells and MSCs respectively, labeled with varying concentrations respectively, labeled with varying concentrations of particles in accordance with the invention.

FIG. 6A is a MRI slice from a 3D data set from a chamber containing live MSC cells.

FIG. 6B is a confocal image of MSC cells labeled with DAPI nuclear counterstaining.

FIG. 7 is a graph showing, T, T₂, and T₂ relaxation time constants for IFP labeled and unlabelled MSC cells.

FIG. 8A is a still frame from a segmented SSFP magnetic resource image of an inferoapical injection of a IFP-labeled MSGs.

FIG. 8B is a T₂ parametric map of the object of the still frame of FIG. 8A.

FIG. 8C is a profile of T₂ values along the dotted white line in FIG. 8B.

FIG. 9A, B, C, and D are 3-dimensional high resolution images of explanted hearts at signed void, day 1, day 4, and day 21 respectively.

FIG. 10A is a confocal fluorescence micrograph of round fluorescent green MSCs with DAPI nuclear counterstaining of surrounding myocardium.

FIG. 10B is a section corresponding to FIG. 10A using differential interference microscopy

FIG. 10C is an overlay of the images of FIGS. 10A and 10B.

FIG. 10D is an endomyocardial engraftment of fluorescent green MSCs with DAPI nuclear counterstaining.

FIG. 10E is a section corresponding to FIG. 10D doing differential interference microscoping.

FIG. 10F is an overlay of the image of FIGS. 10D and 10E.

### DETAILED DESCRIPTION OF THE PREFERRED EMBODIMENT

Particles in accordance with the invention include at least one MRI active material, at least one fluorescent material, and at least one polymer.

MRI Active Material

Particles in accordance with the invention include at least one MRI active material. The MRI active material functions to make the particle, or medium that contains the particle, “visible” or imageable with MRI. Something that is visible to MRI contains a compound or element that can respond to a magnetic field. Examples of MRI active materials include superparamagnetic materials, and paramagnetic materials. For particles that are to be used in vivo a superparamagnetic material is utilized because although it responds to a magnetic field, it displays no residual magnetism. The MRI active material contained within particles of the invention function to make the particle, or the medium that the particle is enclosed in MRI active.

In one embodiment of the invention, the MRI active material may include transition metal oxides, sulfides, silicides and carbides. The MRI active material may also include more than one transition metal. In one embodiment, the MRI active material includes ferrites with the general formula MO.Fe₂O₃ in which M can be Zn, Gd, V, Fe, In, Cu, Co, Mg. Examples of suitable transition metal oxides that can be used in particles of the invention include, but are not limited to: CrO₂, COFe₂O₄, CuFe₂O₄, Dy₃Fe₅O₁₂, DyFeO₃, ErFeO₃, Fe₅Gd₃O₁₂, Fe₅HO₃O₁₂, FeMnNiO₄, Fe₂O₃, γ-Fe₃O₄ (magnetite), α-Fe₃O₄ (hematite), FeLaO₃, MgFe₂O₄, Fe₂MnO₄, MnO₂, Nd₂O₇Ti₂, Al_{0.2}Fe_{1.8}NiO₄, Fe₂Ni_{0.5}O₄Zn_{0.5}, Fe₂Ni_{0.4}Zn_{0.6}, Fe₂Ni_{0.8}Zn_{0.2}, NiO, Fe₂NiO₄, Fe₅O₁₂Sm₃, Ag_{0.5}Fe₁₂La_{0.5}O₁₉, Fe₅O₁₂Y₃, and FeO₃Y. Oxides of two or more of the following metal ions can also be used as MRI active materials in particles of the invention: Al(+3), Ti(+4), V(+3), Mn(+2), Co(+2), Ni(+2), Mo(+5), Pd(+3), Ag(+1), Cd(+2), Gd(+3), Tb(+3), Dy(+3), Er(+3), Tm(+3) and Hg(+1). Because these non-ferrites are often colored, they can also make the particles “imageable” by spectrophotometric techniques.

In one embodiment, one or more materials that display superparamagnetic properties are utilized in a particle in accordance with the invention. Examples of materials that display superparamagnetic properties include but are not limited to magnetite (γ-Fe₃O₄), and hematite (α-Fe₃O₄).

In one embodiment of the invention, the MRI active material includes crystals of magnetite. The size of the magnetite crystals depend at least in part on the size of the final particle and the magnitude of the magnetic signal that is desired. In one embodiment, the diameter of the magnetite crystals ranges from about 1 to 20 nm.

Particles of the invention utilizing magnetite as the MRI active material generally have from about 40 to 65% magnetite. In one embodiment, particles of the invention have about 60 to 65% magnetite. In yet another embodiment, particles of the invention have about 63% magnetite.

Fluorescent Material

Particles in accordance with the invention also contain at least one fluorescent material. The fluorescent material functions to make the particle, or the medium that contains the particle, “visible” to fluorescent detecting techniques. Examples of fluorescent detecting techniques include, but are not limited to, flow cytometry, fluorescence microscopy, and confocal microscopy. A fluorescent material is one that emits radiation, with or without a change in frequency, when it absorbs radiation of a specific wavelength or wavelength range.

Generally, fluorescent compounds contain aromatic functionality, aliphatic and alicyclic carbonyl structures, or highly conjugated double-bond structures. There are a number of structural properties and characteristics that can affect the fluorescent properties of a molecule. Examples of fluorescent materials include, but are not limited to fluorescein isothiocyanate, rhodamine-isothiocyanate, dragon green, phycoerythrin, 7-amino-4-methylcourmarine-3-acetic acid, bis-benzimide, indocarbocyanine, indodicarbocyanine, lissamine rhodamine B-sulfonyl hydrazine, lucifer yellow CH, phycocyanine, Texas red, and allophycocyanine.

In one embodiment of the invention, fluorescein, with the structure given below is used as the fluorescent material.

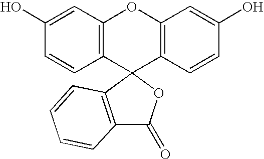

In one embodiment of the invention a derivative of fluorescein, fluorescein isothiocyanate (FITC) is utilized as the fluorescent material. The structure of FITC is given below.

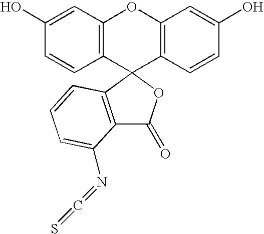

In general, if more fluorescent material is included in a particle of the invention, the greater the detectable fluorescent signal will be. The amount of the fluorescent active material that is incorporated into a particle of the invention depends at least in part on the fluorescent signal, the detectable levels of the fluorescent material and the desired minimum detection levels of the medium containing the particles. Because different fluorescent materials have different strengths, the concentration of the fluorescent material in the particle can vary. In one embodiment, enough of the fluorescent material is added to the particle of the invention so that cells labelled with the particles have a mean fluorescence with an intensity two logs higher than unlabeled cells. In yet another embodiment, enough fluorescent material is added to the particle of the invention so that cells labelled with the particles have a mean fluorescence with an intensity three logs higher than unlabelled cells.

Polymer

Particles in accordance with the invention also contain at least one polymer. The at least one polymer functions to provide structure to the particle, and maintain the MRI active material and the fluorescent material together.

In one embodiment, the at least one polymer used in particles in accordance with the invention is biocompatible. Biocompatible means compatibility with living tissue or a living system by not being toxic or injurious and not causing immunological rejection. In one embodiment of the invention, the polymer or polymers used in particles of the invention either bear, or are capable of being functionalized to bear —COOH or —NH₂ groups. Such groups can be advantageous in that it can allow biomolecules to be attached to the surface of particles in accordance with the invention.

An example of a polymer that can be utilized in particles of the invention include polymeric vinyl alcohol, or polyvinyl alcohol (PVOH), which is a polyhydroxy polymer having a polymethylene backbone with pendent hydroxy groups. PVOH is a water soluble synthetic resin.

Solutions of polyvinyl alcohol in water can be made with large quantities of lower alcoholic cosolvents and salt cosolutes. Polyvinyl alcohol can react with aldehydes to form acetals, can be reacted with acrylonitrile to form cyanoethyl groups, and can be reacted with ethylene and propylene oxide to form hydroxy alkaline groups. Polyvinyl alcohols can be readily crosslinked and can be borated to effect gelation.

Polymers for use in particles in accordance with the invention may also result from the polymerization or copolymerization of monomeric alpha, beta unsaturated carboxylic acid or monomeric esters of alpha, beta unsaturated carboxylic acid. Suitable monomers include those containing a carboxylic acid or carboxylate group as a functional group and include a vinyl monomer having a free carboxylic acid or carboxylate functional group.

In one embodiment, the polymer results from a carboxylic acid containing monomers comprising alpha, beta unsaturated carboxylic acids including methacrylic acid, acrylic acid, itaconic acid, iconatic acid, aconitic acid, cinnamic acid, crotonic acid, mesaconic acid, carboxyethyl acrylic acid, maleic acid, fumaric acid, 3-acrylamidopropanesulfonic acid, 2-acrylamido-2-methylpropanesulfonic acid, 3-acryloyloxypropanesulfonic acid, and the like. The carboxylic acid functional copolymer can contain other ethylenically unsaturated monomers compatible with the ethylenically unsaturated carboxylic acid containing monomers disclosed above. Such monomers include ethylene, propylene, isobutylene, vinyl chloride, vinyl acetate, styrene, chlorostyrene, and the like. Further, the polymers can contain hydrophilic ethylenically unsaturated monomers having amino groups, hydroxyl groups, ether groups, ester groups, and others.

Alternatively the carboxylic acid functional polymer can be a polysaccharide having pendent carboxylic acid groups. Examples of such polysaccharide carboxylic acid functional polymers include carboxymethyl cellulose and carboxylethyl cellulose, carboxymethyl starch and carboxyethyl starch, alginic acid and alginic acid derivatives, pectic acid or similar natural and synthetic carboxylic acid derivatives of a polysaccharide. Also useful in the synthesis of an acrylic copolymer for use in particles of the invention are esters of alpha, beta unsaturated carboxylic acid such as those mentioned above.

The alkyl esters may be selected from higher alkyl esters such as those of about 5-22 carbon atoms. Examples of C₅₋₂₂ compounds include hexyl, octyl, ethyl (hexyl), isodecyl, and lauryl, acrylates, and methacrylates and itaconates. Alkyl esters having branched as opposed to straight chain moieties are also useful as copolymers for use in particles of the invention.

An additional family of monomers which has been found useful in producing polymers for use in particles of the invention are polymeric ethylene oxide resins. Generally ethylene oxide has the formula: H(OCH₂CH₂)ₙOH.

Polyethylene oxides are generally clear viscous liquids, or depending on molecular weight and moles of ethylene oxide, white solids which dissolve in water, forming transparent solutions. Polyethylene oxide is soluble in many organic solvents and readily soluble in aromatic hydrocarbons while only slightly soluble in aliphatic hydrocarbons. Polyethylene oxides are generally classified not only by moles of ethylene oxide present within the composition, but also by molecular weight.

In one embodiment of the invention particles of the invention containing polystyrene as the polymer are utilized.

Particles

In one embodiment of the invention, particles in accordance with the invention are uniform throughout. Such particles can be made using a number of methods. One example of a method of forming a particle of the invention includes combining the MRI active material, the fluorescent material, and the at least one polymer (or the prescursors of any of these components) in a liquid mixture, and including another liquid, usually water to form a suspension. The suspension can then be treated in a number of ways to make stable suspensions of particles. These treatment methods include, but are not limited to, mechanical treatment methods such as heat, vibration, irradiation, and sonication; chemical treatments such as pH modification; or a combination thereof.

Another example of a method of making particles of the invention is to prepare a mixture of the MRI active material precursors in water, pH adjust the solution to form the precursors into the active MRI material, washing the particles, and depositing the polymer or polymer precursors on the particles and if necessary treating the precursors to form the polymer.

Yet another example of a method of making the particles of the invention begins by adding the MRI active component, such as the iron oxide particles, and the fluorescent material, such as the FITC, to monomer units of the polymer of the particle. In such an embodiment, the iron oxide particles and FITC are incorporated into the particle by being trapped in the polymer matrix. In this embodiment, the distribution of the iron oxide particles, the FITC, and the polymer is generally homogeneous throughout.

Another example includes making the particles in accordance with the invention by adding the MRI active material and the fluorescent material to a polymer particle by swelling the cage matrix of polymer particle with an organic solvent, adding the fluorescent material and as much of the MRI active material as possible. Then the cage matrix is shrunk back to an average size of 0.9 μm by removing the solvent and replacing it with a sterile aqueous solution.

In another embodiment of the invention, the MRI active material can be encapsulated by a polymer matrix containing the fluorescent material. In yet another embodiment, the MRI active material can be encapsulated by a polymer matrix which has the fluorescent material attached to the outside thereof.

Examples of particles which can have a fluorescent material incorporated in to make a particle of the invention include, but are not limited to, those available from Bangs Laboratories, Inc. (Fishers, Ind,). Specifically, such particles include estapor® SuperParaMagnetic Microspheres, catalog code MCO4N, MCO5N, MCO0N, and MCEO3N for example.

Particles of the invention generally have diameters from about 0.35 to about 2.5 μm. In one embodiment, particles of the invention have diameters of from about 0.75 to about 1.25 μm. In yet another embodiment, particles of the invention have diameters of about 0.9 μm.

Methods of Using Particles of the Invention

The invention also provides methods of tracking at least one cell in an environment that includes the steps of labeling the cell with a particle that includes at least one MRI active material, at least one fluorescent material, and at least one polymer, and monitoring the labeled cell within the environment by monitoring the particle using at least one technique chosen from the group consisting of: magnetic resonance imaging (MRI), flow cytometry, fluorescent techniques, and microscopy.

Methods of the invention can be used to track any type of cell. Examples of cells that can be tracked include, but are not limited to, hematopoietic stem cells, lymphocytes, mesenchymal stem (MSCs), muscle satellite cells, embryonic stem cells, neural stem cells. The cell to be tracked need only be able to be labeled with the particle in accordance with the invention. Tracking a cell includes, but is not limited to, locating and monitoring the cell within the environment within which it exists. Methods of the invention can be used to track cells in vivo or in vitro.

Generally, labeling a cell with a particle in accordance with the invention includes the uptake of the particle by the cell. The particle can be taken into the cell through a number of different processes, including but not limited to, phagocytosis, endocytosis or microinjection. In one embodiment of the invention, the mechanism of the incorporation of particles in accordance with the invention into MSCs includes nonspecific phagocytosis during proliferation. The structure of the particle itself can enhance the uptake of the particle by the cell. For example, the surface of the particle can be modified so that it is a better candidate for phagocytosis or endocytosis. One example of a surface modification includes attachment of a specific antibody to a cell surface protein that may promote endocytosis. In one embodiment of the invention, particles in accordance with the invention are taken up very rapidly and with high efficiency into the cells, with no apparent toxicity or impact on bioactivity, despite dense loading of the cells with the particles.

Methods of the invention include at least one step of monitoring the particle or the cell containing the particle using at least one of a number of methods. Monitoring the particle can be accomplished by using magnetic resonance imaging (MRI), flow cytometry, fluorescent techniques, and microscopy.

Particles in accordance with the invention, and methods of the invention can offer several potential advantages for certain applications compared to previously described agents. Particles in accordance with the invention can be utilized to detect the presence of single cells at lower resolutions (200 μm) than are commonly used. Furthermore, particles in accordance with the invention can create a much greater magnetic moment within individual cells, increasing the likelihood that in vivo imaging of single cells or very small numbers of cells is possible. This can be particularly important for studying hematopoietic stem cell homing to the marrow, since very small numbers of highly purified HSCs are used to analyze the determinants of engraftment.

Methods of the invention also offer a minimum detectable quantity (10² cells/injection) of cells necessary for detection using conventional cardiac MRI on a commercially-available scanner. This is at least one order of magnitude lower than projected injection of cellular agents and should accommodate “tracer” quantities of labeled MSCs admixed with unlabeled MSCs.

### WORKING EXAMPLES

The following examples provide non-limiting illustrations of the invention.

### Example 1

### Magnetic Susceptibility of Particles of the Invention

Superparamagnetic di-vinyl benzene inert polymer particles containing FITC were manufactured by Bangs Laboratories, Inc. (Fishers, Ind.). The average size of the particles was about is 0.9 μm. The particles contained 63.4% magnetite iron-oxide component as well as fluorescein-5-isothiocyanate (FITC) analogue component trapped within the polymer matrix. Both of the components were added to the particle by swelling the cage matrix with an organic solvent, adding the fluorophore and as much iron oxide as possible, and then shrinking the matrix back to the average size of 0.9 micron by removing the solvent and replacing it with a sterile aqueous solution. Particles were prepared for target cell exposure by washing with phosphate buffered saline (PBS). The particles were then immobilized using a magnet and resuspended at a concentration of 1% weight per volume sterile PBS. The iron content of this suspension was approximately 4.56 mg/ml.

The particle suspension was stored at 4° C. and gently resuspended prior to labeling procedures. In vitro comparisons on the effects of the size of particles on MR images were done with dilutions of Feridex™ particles (Berlex Laboratories, Wayne, N.J.). Feridex™ particles are an FDA-approved iron oxide nanoparticle that consists of a single nanocrystal of iron oxide with a diameter of about 7-10 nm diameter coated with dextran.

The particles in accordance with the invention and the Feridex™ particles were used at iron concentrations of 1.0, 0.5, 0.2, 0.1, 0.01 mM in 2% agarose in small culture tubes. The culture tubes were then themselves embedded in 2% agarose in a small beaker to ameliorate susceptibility effects around the tubes. The sample ensemble was placed inside a 3.5 cm diameter, 8.5 cm long Bruker birdcage coil. 2D MRI was performed with the following imaging parameters: TE=7 ms, TR=4000 ms, FOV=6×6 cm, slice thickness=2 mm, matrix=512×512, NEX=1, BW=101 kHz and total imaging time=34 min. These parameters yielded a pixel size of 117×117×2000 microns.

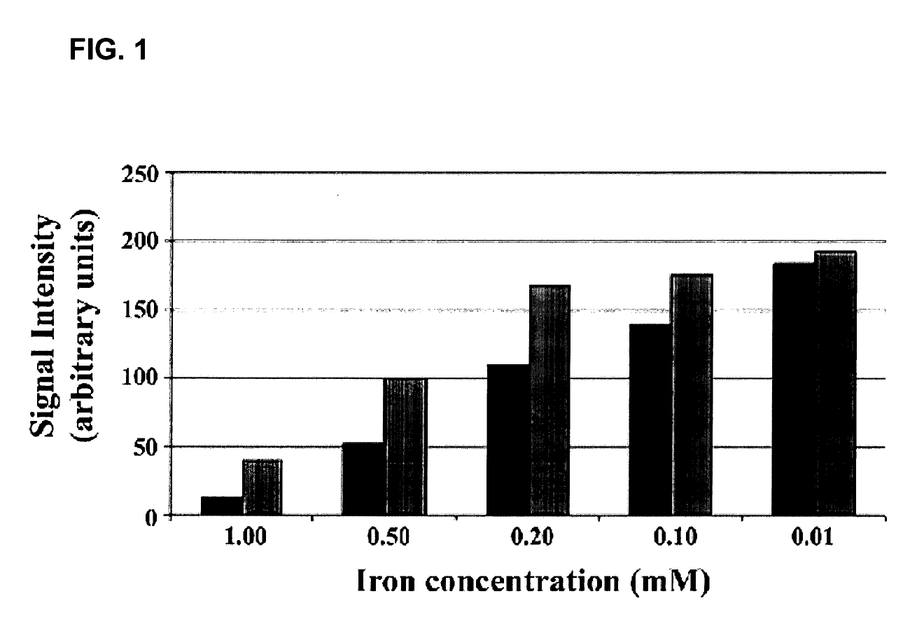

FIG. 1 shows the final concentration (x-axis) versus the signal intensities of T₂* weighted images (y-axis) of dilutions of Feridex™ 10 nm particles (light gray bars) and particles of the invention (relative size of magnetite core is 760 nm-dark gray bars) in 2% agarose gels.

As seen in FIG. 1, with equal iron content, the particles of the invention yielded darker images under T₂* weighted imaging conditions than did the Feridex™ particles. The relaxation enhancement, as a percent difference, of the two different particles is generally greatest with at higher iron concentration. However the highest magnitude difference occurred with an intermediate concentration. The amplification effects of particles of the invention with respect to the Feridex™ particles are clearly evident down to concentrations of 100 μM iron.

As seen in the direct comparison data, when normalized to iron content, the particles of the invention greatly decrease T₂* and enhance the susceptibility effect in the images. While the total iron content of the samples containing the particles of the invention and the comparison particles were kept equal, the sizes of the particles were different. Feridex™ has a 10 nm core, while the effective magnetite core size of the particles of the invention used for this experiment was 760 nm. This results in nearly 5000-fold fewer particles of the invention than Feridex™ particles in each comparison tube. If the magnetic resonant effects were normalized to the number of particles the disparity would be much greater.

### Example 2

### Uptake of Particles of the Invention By Cells Cell Culture and Labeling

Purified CD34+ cells were obtained using an Isolex™ 300 Magnetic Cell Separator (Baxter, Irvine, Calif.). The mean purity was 85-95% CD34+. CD34+ cells were cultured in stem cell media (SCM), consisting of Dulbecco's Modification of Eagle's Medium (DMEM), 10% fetal bovine serum (FBS), 4 mM L-Glutamine, 50 mg/ml of penicillin and streptomyocin, and 100 ng/ml each of recombinant human Flt-3 ligand (Immunex, Seattle, Wash.), stem cell factor (SCF) (Amgen, Thousand Oaks, Calif.), and megakaryocyte growth and development factor (MGDF) (Amgen). CD34+ cells were seeded at a concentration of 500,000 cells/well in 200 μl of SCM in a 96-well plate, and the particle suspension was added to each well. The cells were cultured overnight at 37° in 5% CO₂. The next morning, cells were removed from the wells, washed three times via centrifugation at 1,500 rpm. For cell labeling, 1 μl/ml fluorescent particle suspension was added to the wells and the cells were incubated at 37° C. for 18 hours. The cells were collected, washed and resuspended on a chamber slide for microscopy.

Peripheral blood progenitor cells were obtained by aphaeresis from normal volunteers entered on an Institutional Review Board-approved protocol, following five subcutaneous injections of 10 g/kg/day Granulocyte Colony Stimulating Factor (G-CSF).

Primary porcine bone marrow mesenchymal stem cells (MSCs) were derived from bone marrow aspirates obtained from healthy adult farm swine. Aspirates were diluted with 2 volumes of PBS, washed, and the mononuclear cells isolated by density gradient centrifugation (Ficoll-Paque™; Amersham-Biosciences Corp, Piscataway, N.J.). Recovered cells were washed twice in PBS and resuspended in mesenchymal stem cell growth medium (MSCGM) (Poeitics, Biowhittaker, Walkersville, Md.) supplemented with MSCGM bullet kits (Poeitics, Biowhittaker). Cells were then seeded at a concentration of 1000 cells/mm² in supplemented MSCGM. After 5 days non-adherent cells were removed and adherent colonies expanded further in culture. The MSCs were labeled by adding iron oxide particle suspension (10 μl/ml) to the non-confluent MSCs and incubated overnight at 37° C. in 5% CO₂. Excess particles were removed by washing with PBS.

All imaging experiments were conducted using a Zeiss LSM 510 confocal microscope (Car Zeiss, Inc. Thornwood, N.Y.) and a C-Apochromat 63×, 1.2 N.A. lens (Carl Zeiss, Inc.). FITC and Cell Tracker Orange were imaged sequentially using 488 and 543 nm excitation light and 505-530 and 560-615 nm band-pass filters respectively. The pinholes on the emission channels were set on all experiments to produce an optical slice thickness of 1.5 mM. Images were acquired sequentially and band-pass emission filters were used to avoid spectral bleed-through between the imaging channels.

To image the mesenchymal stem cells (MSCs), the cells were seeded at varying concentrations into 2 well glass bottomed chamber slides after washing off non-endocytosed particles. To confirm endosomal uptake CM-DiI (Molecular Probes) and DAPI (Molecular Probes) for nuclear counterstain were added as per manufacturer's instructions.

Cells used for confocal microscopy were incubated with or without the particle of the invention and other cellular stains that contrast with the FITC green color wavelength. Cells that were incubated in Cell Tracker Orange (Molecular Probes, Eugene, Oreg.) were first washed with warm PBS and then adhered to a poly-L-lysine coverslip for 30 minutes at 37EC, 5% CO₂. Following incubation, cells were gently rinsed with warm PBS three times. The coverslip was then covered with an adhesive confocal coverwell (Grace Bio-Labs, Bend, Oreg.) and filled with warm PBS.

Electron Microscopy

One million cells were initially fixed with 1.25% glutaraldehyde in 0.1M cacodylate buffer (containing 0.1M sodium cacodylate trihydrate, 0.4 mL hydrochloric acid and 0.05%, calcium chloride at a pH of 7.4 for a total of one liter) at 4° C. overnight. After washing in Sabatini solution (0.1M cacodylate buffer containing sucrose and calcium chloride) the cells were post-fixed in 1% osmium tetroxide, dehydrated through ascending alcohol and propylene oxide, and embedded in SCI Poxy 812 (Energy Beam Sciences, Agawam, Mass.). Ultrathin sections were cut with a Leica Ultracut UCT (Leica Microsystems, Inc., Bannockburn, Ill.), stained with uranyl acetate and lead citrate, and examined with a JEOL 1200 EXII transmission electron microscope (JEOL, Peabody, Mass.).

Results

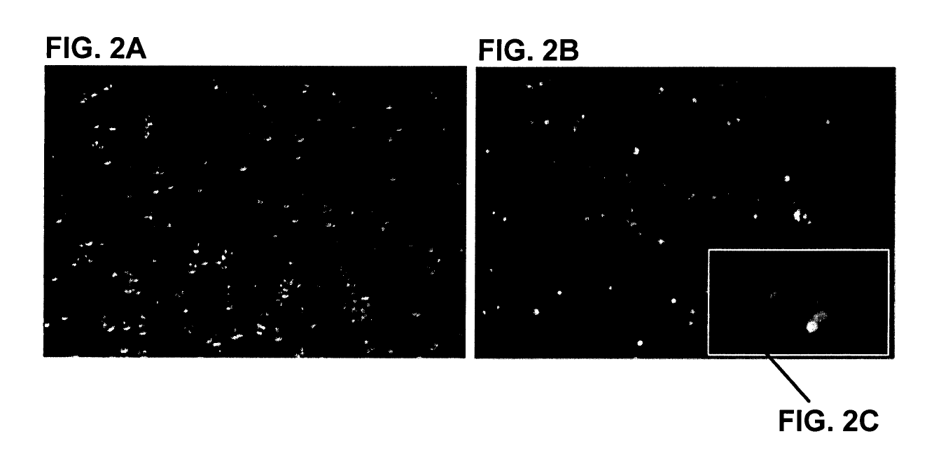

CD34+ cells were exposed to various concentrations of particles in accordance with the invention. CD34+ primary hematopoietic cell populations include primitive lineage-committed progenitor cells and true long-term repopulating stem cells able to fully reconstitute patients following myeloablation and autologous or allogeneic stem cell transplantation. These cells were chosen as a representative non-adherent cellular target. Incubation with particles in accordance with the invention for 12-18 hours at concentrations down to 2.5 μl per ml resulted in relatively homogeneous labeling of greater than 90% of these cells. FIG. 2 shows a light microscopy images of the cells, showing a uniform population of primitive hematopoietic progenitors with no evidence of toxicity and some cells undergoing active cell division. FIG. 2B is a fluorescent micrograph of the same field, showing that >90% of the cells fluoresce green, with relatively homogeneous intensity. FIG. 2C is a higher power view of a fluorescent cell in the midst of mitosis, with segregation of the label occurring to both daughter cells. Labeling periods as brief as one hour resulted in similar labeling efficiency and intensity, indicating rapid uptake. Primary human peripheral blood lymphocytes could also be labeled with similar efficiencies (data not shown).

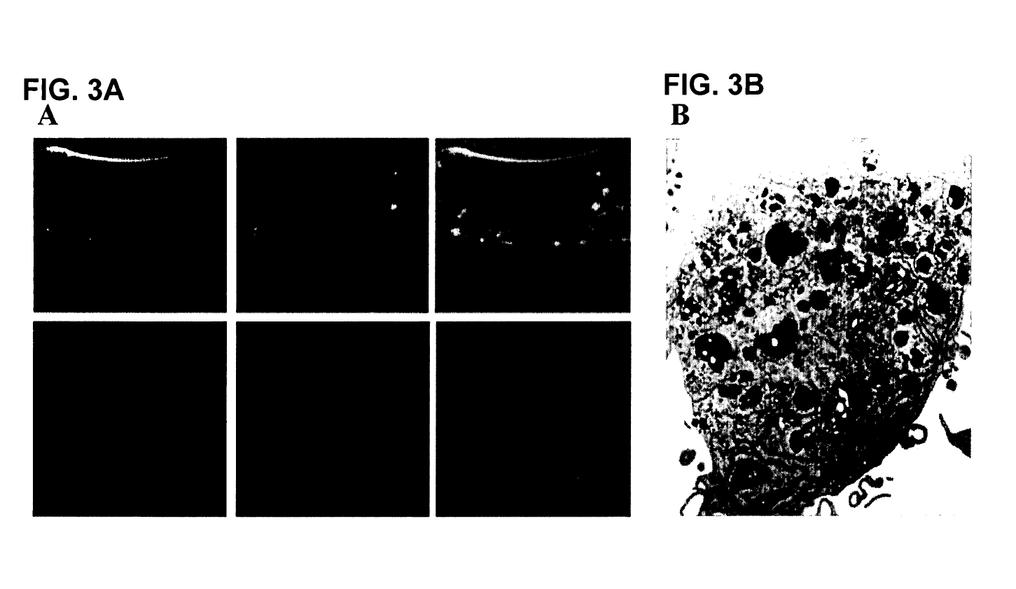

FIG. 3A reveals through confocal fluorescence microscopy (FIG. 3A) revealed a cytoplasmic granular distribution of the particles within the CD34+ cells. The far left panel shows phase contrast images of the CD34+ cells, the upper center panel shows the green fluorescence of the cells, and the upper far right panel overlays the images. The lower far left panel shows green fluorescence from the iron oxide particles within the cell. The lower center panel shows Cell Tracker orange fluorescence of cytoplasm of viable cells, and the lower far right panel shows both, demonstrating a granular distribution of the particle fluorescence throughout the cytoplasm of the cell. FIG. 3B shows electron microscopy showed profuse uptake of the large iron oxide particles, with encasement of the parties within membrane-bound organelles.

To further demonstrate the labeling efficiency of the particles, porcine primary bone marrow-derived mesenchymal stem cells (MSC) were chosen as a representative adherent cell population. A subconfluent monolayer of primary porcine marrow MSCs were exposed to 10 μl/ml of the iron oxide fluorescent microparticles overnight. Excess particles were washed off with PBS. Overnight exposure of MSCs to 10 μl/ml of particles in accordance with the invention resulted in efficient labeling with localization at high densities in perinuclear cytoplasm.

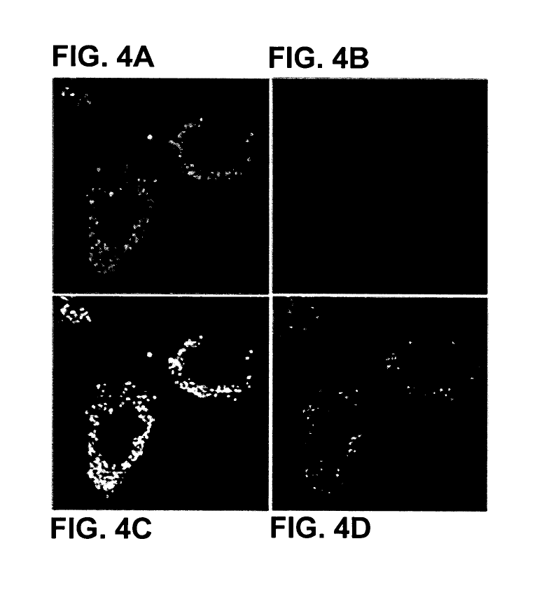

FIG. 4 shows comparison of particle labeling with CM-DiI labeling to confirm endosomal uptake.

FIG. 4A shows green fluorescence of the particles. FIG. 4B shows red fluorescence of endosomal marker CM-DiI. FIG. 4C shows co-localization of the two colors confirming endosomal particle uptake. FIG. 4D shows a Nomarski optics view revealing the outlines of the fibroblastic cells and the iron particles clearly clustered in perinuclear organelles.

### Example 3

### Preservation and Proliferation of Differentiation Hematopoietic Colony Culture

Following particle exposure at a concentration of 0-25 μl/ml for 18 hours, CD34+ cells were plated in duplicate in standard semi-solid methylcellulose hematopoietic progenitor culture media (human MethoCult+GF) (Stem Cell Technologies, Vancouver, BC) at concentrations from 5×10²-10⁴/ml. These culture plates were incubated at 37° C. in 5% CO₂. Colonies consisting of >50 cells were identified and enumerated 12-14 days later.

Proliferation Assay:

Labeled MSC viability was assessed by trypan blue exclusion (data not shown) and in vitro proliferation using a modified MTT assay (3-[4,5-dimethylthiazol-2-yl]-2,5-diphenyl tetrazolium bromide) (Roche Diagnostics, Indianapolis, Ind.). Briefly 1000 MSCs/well of a 24 well plate were seeded at early passage (P3-P5) and growth curves established after overnight labeling with a range of particle concentrations (0.1-50 μl/ml) of particles in accordance with the invention. Particles were either removed by repeated washing and replaced with fresh media or left in the media for the duration of the assay. Quadruplicate samples were assayed at each time point and the experiment repeated in triplicate.

Results

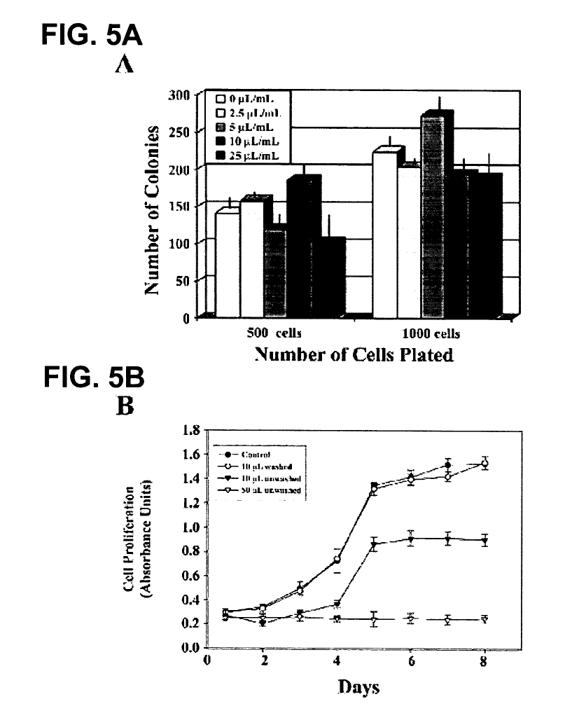

FIG. 5A depicts the growth of CD34+ cells labeled with particles in accordance with the invention. CD34+ human progenitor cells were exposed to a range of concentrations of particle in accordance with the invention for 18 hours and then plated in methylcellulose. 10-12 days later, macroscopic colonies (CFU) were enumerated. The individual bars of FIG. 5A gives the concentration of particle added following plating of either 500 or 1000 cells, and the y axis the number of CFU present. There was no difference in the average size or composition of the individual colonies. The colony number data is derived from three independent experiments using different CD34+ cell donors. CFU number and proliferative capacity were not significantly altered, nor was the lineage commitment of each individual progenitor, since the colonies were of normal size and there was no change in distribution of myeloid and erythroid colonies (FIG. 5A). Even after the 8-10 cell divisions on average required to form a CFU, the label appeared to distribute to each cell in the colony, as assessed by fluorescent microscopy.

FIG. 5B depicts the growth of MSCs labeled with particles in accordance with the invention. MSCs were exposed to a range of particle concentrations and growth assessed using an MTT assay. No effect on cell proliferation was observed after overnight labeling with 1 or 10 μl/ml. Proliferation was mildly impaired if particles were not removed from the media for the duration of the growth assay. The y axis shows cell proliferation in absorbance units at 560 m and the x axis days in culture. MSCs were not affected by 18 hour exposure to a range of bead concentrations from 0.1 μl/ml-50 μl/ml. Cell proliferation was marginally reduced if beads at effective labeling concentration were not removed from the media for the duration of the growth assay. An adipogenic differentiation assay also demonstrated no effect on MSC differentiation in labeled and unlabeled cells (data not shown). The segregation of the particles into membrane-bound cytoplasmic perinuclear endosomes appears to allow incredibly dense iron particle loading of cells, without serious impact on other cellular processes.

### Example 4

### Single Cell MRI

Cellular MRI:

Cellular imaging was performed on an 11.7 T Bruker Advance Spectrometer (Billerica, Mass.). Two chamber culture dishes with live MSCs culture media were placed inside a 3.5 cm diameter, 8.5 cm long Bruker birdcage coil. After appropriate image localization scans, a high resolution, 3D gradient echo pulse sequence was run on the entire sample. Imaging parameters were as follows: TE=4 ms, TR=200 ms, FOV=5×3×0.2 cm, matrix=512×512×40, NEX=4, BW=101 kHz, total imaging time=4.5 hours. These parameters yielded a voxel size roughly of 100×50×50 microns.

Results.

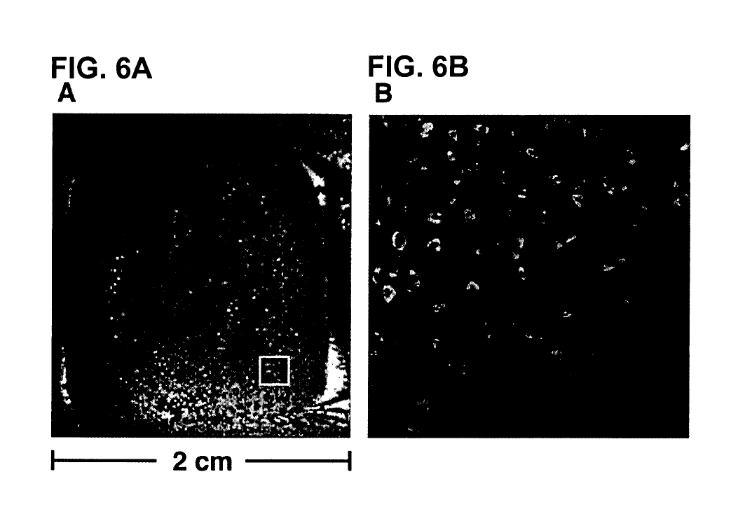

Single cells could be detected in vitro using MRI with a signal void corresponding to the position of each cell. FIG. 6A shows one slice from a 3D data set from a chamber containing live, labeled MSCs in culture media. Single cells are present where there are dark spots in the dish, owing to the susceptibility effect of the endocytosed particles. Some spots are darker and larger than others, owing to both partial volume effects inherent in the imaging parameters and differential amounts of label incorporated into each cell.

FIG. 6B is a representative confocal image showing labeled cells with DAPI nuclear counterstaining shows the efficiency of MSC labeling is almost 100%. The combination of very efficient uptake and large particle size results in greatly increased resolution on MRI. The greatest magnitude of changes involved T₂*, with much less marked T₁ effects. The lack of a significant T₁ effect is expected as the iron oxide core is shielded from the solvent.

### Example 5

### In Vivo Animal Studies

Animal protocols were approved by the NHLBI Annual Care and Use Committee. Myocardial infarctions were created in three to six month old Hanford mini-swine (30-40 kg) by transcatheter occlusion of the left anterior descending coronary artery using platinum coils (VortX, Boston Scientific Target, Cork, Ireland). Allogenic MSCs from Yorkshire swine were injected under X-ray guidance percutaneously using steerable, biocompatible, coaxial guiding catheters to position a spring-actuated 27G needle (Stilletto™, provided by Boston Scientific Molecular Interventions, Natick, Mass.). Two to five injections were performed in each animal, and needle dead-space was cleared before each needle retraction. Five animals were survived for up to 21 days to allow serial MRI. Explanted hearts were snap-frozen for -immediate histological examination for the presence of IFP-labeled MSCs. Frozen sections were fixed with cold methanol, washed with PBS and mounted with DAPI containing mounting medium (Vectashield, Vector Laboratories). The images were taken by using Leica TCS-SP upright confocal microscope. Alternatively hearts were retrograde-perfused with 4% formaldehyde for high-resolution overnight 3-dimensional MRI (fast gradient echo, voxel size x x x x 1 mm, x x x(matrix), field of view 12 cm, repetition time 10 ms, echo time 3.3 mS, flip angle xxo, 1.5T CV/i and head coil, General Electric, Waukesha, Wis.) after epicardial placement of gadolinium-filled fiducial marker beads. Hearts were then sectioned to examine the presence of IFP-loaded cells.

Relaxometry and In Vivo MRI

MRI contrast characteristics of IFP-labeled MSCs were studied by measuring spin-lattice (T1) and spin-spin (T2) relaxation time constants in cell suspension, ex vivo cell injections, and after in vivo endomyocardial injection. Dual-labeled MSCs were washed, trypsinized and counted using a hemocytometer and resuspended in 1 mL of 1% low melting point agarose as previously described (Schulze, E. et al. Invest Radiol 30, 604-610 (1995)) at a range of cell densities from 10² to 10⁷ cells/mL. The gel suspensions were then transferred to 24 well plates and imaged using a 1.5T MRI (Sonata, Siemens, Erlangen, Germany) and spine phased array coil. To simulate relaxometry after endomyocardial injection, IFP-labeled MSCs were suspended at a range of concentrations (10⁴ to 10⁶) in 150 μL injection volumes of PBS and injected through 27G needles into uniform 10 mm sections of fresh porcine myocardium before MRI. Matching non IFP-labeled MSCs served as a control. The T₁, T₂ and T₂*-relaxation rates of the IFPs within cell suspensions and injected tissue were measured at 37° C. T₁ was measured using steady state free precession (SSFP) inversion recovery pulse sequences and multiple inversion times (196-1000 ms); T₂, by fast spin echo with multiple effective echo times (3.4-90 ms); T₂*, by fast gradient echo (FGRE) with multiple echo times (2.6-60 ms). The field of view was 360 mm which gave voxel sizes of 1.4×1.9×6.0 mm. Voxel-by-voxel relaxation curves were generated and fitted using a non-linear least-squares algorithm (MatLab v6.1, Mathworks, Natick, Mass.).

For in vivo MRI, serial dilutions (10⁴-10⁶) of IFP-labeled MSCs were suspended in 150 μL injection volumes of PBS and injected percutaneously into infarcted and normal myocardium segments under fluoroscopic guidance (Multistar, Siemens). MRI was performed at multiple time points using both SSFP and FGRE pulse sequences. In vivo T₂* relaxation rates were determine using gated FGRE with multiple echo times (3-20 ms). Signal-to-noise (SNR) and contrast-to-noise (CNR) ratios were measured for normal, infarcted, and injected myocardium according to the relation (CNR-SI_{myo}-SI_{IFP}]/SDₙₒᵢₛₑ), where SI_{myo} represents the signal intensity (in arbitrary units) of normal myocardium, SI_{IFP} represents the signal intensity of IFP-labeled cell injection sites, and SDₙₒᵢₛₑ represents the standard deviation of background noise.

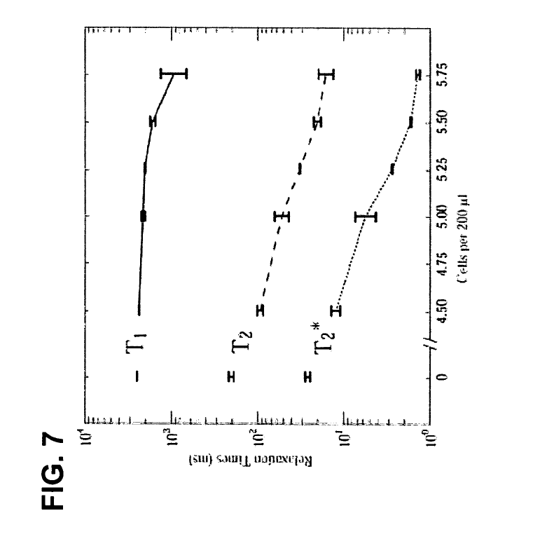

FIG. 7 shows T₁, T₂ and T₂* relaxation time constants for IFP-labeled and unlabeled MSC suspension in agar at a range of cell concentrations. The particles imparted little T₁ or T₂ contrast. Denser cell preparations imparted shorter T2*.

These relaxation times were also measured at ex vivo injections into freshly explanted normal myocardium using the highest-delivered cell host (10⁶ cells). The T2* of IFP-labeled MSCs was significantly different from unlabeled cells and from normal myocardium.

The T₂* values for in vivo intramyocardial injections of 10⁵/150 μl IFP-labeled MSCs and normal myocardium are 9 ms versus 27 ms.

TABLE 1

| Relaxation | Cell | Cell | | |
|---|---|---|---|---|
| time | Suspension | Suspension | Ex vivo | |
| constants | unlabeled cells | labeled cells | injection | Normal |
| (ms) | (10⁵/150 μL) | (10⁵/150 μL) | (10⁵/150 μL) | myocardium |
| T₁ | 2500 ± 23 | 2202 ± 90 | 1238 | 1150 ± 75 |
| T₂ | 205 ± 14.8 | 53.1 ± 11.2 | 95.0 | 93.0 ± 4.0 |
| T₂* | 24 ± 2.2 | 5.7 ± 1.8 | 6.0 | 24.8 ± 2.0 |

Serial In Vivo MR Imaging

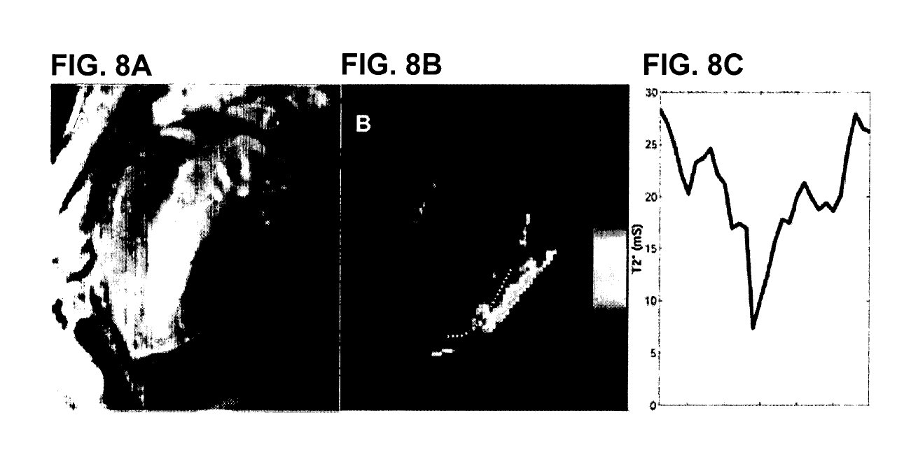

FIG. 8 shows a typical inferoapical injection of IFP-labeled MSCs in still frame from a segmented SSFP magnetic resonance image. The injection appears as a dark spot in mid-myocardium. FIG. 8B shows a T2* parametric map of the same slice, which shows the lowest T2* in the region of the injection. The posterobasal epicardial surface also shows a typical susceptibility SSFP artifact (lower T2* values) commonly attributed to adjacent diaphragmatic surfaces. FIGS. 8C shows a profile of T2* values along the dotted white line from the middle panel.

Ex Vivo Imaging

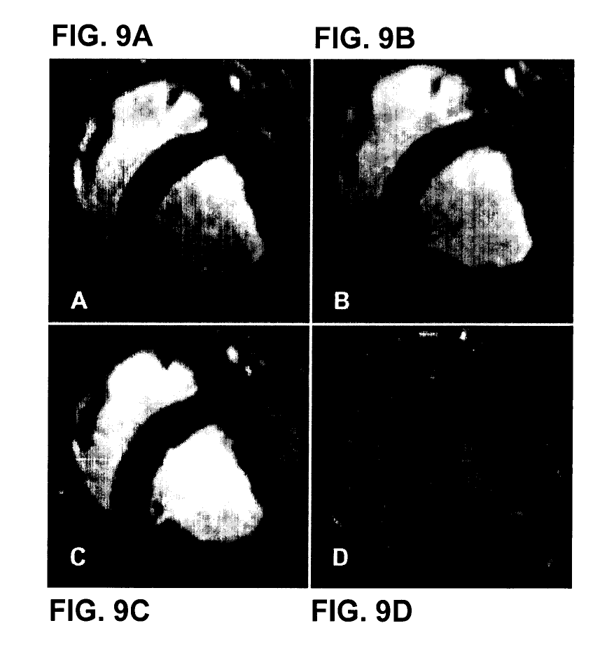

Explanted hearts(n=3) which underwent 3-dimensional high resolution MRI at the signal void (FIG. 9A), day 1 (FIG. 9B), day 4 (FIG. 9C), and day 21 (FIG. 9D) further confirmed injectate position relative to extraanatomic markers.

Corresponding short axis view of explanted heart following formalin retrograde perfusion and high resolution overnight MRI showing signal void. Labeled cells could be identified after injection into both normal and infarcted myocardium. A magnetic susceptibility artifact was seen as a signal void (“black hole”) corresponding to the injection sites. Injection volumes of 150 μL generated signal void volumes of 0.36±0.38 cm³. The contrast to noise ratio between normal myocardium and the signal void created by the injected IFT was 8.9. Serial MRI studies were conducted for up to 3 weeks. Injections of 1- 2×10⁶ MSCs were seen serially although injections of 1×10⁵ MSCs were visualized less reproducibly.

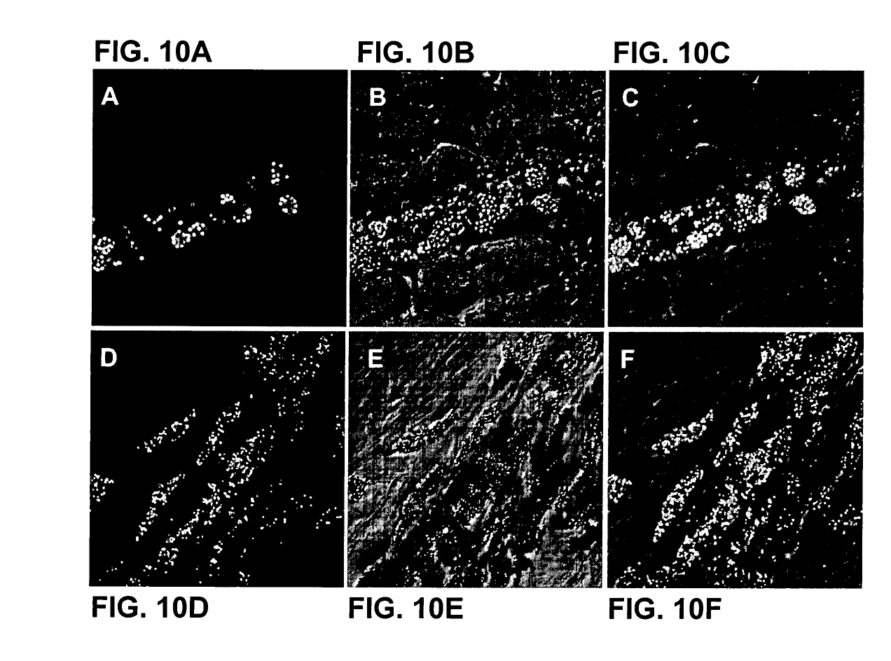

IFP-labeled MSCs were subsequently identified at the corresponding injection sites by histopathology using both confocal fluorescence microscopy and differential interference contrast (FIG. 10). IFP-labeled MSCs are recovered within myocardium immediately after injection and appear rounded (FIGS. 10A-C). After 3 weeks IFP-labeled MSCs could still be detected but appeared more elongated (FIGS. 10D-F).

The above specification, examples and data provide a complete description of the manufacture and use of the composition of the invention. Since many embodiments of the invention can be made without departing from the spirit and scope of the invention, the invention resides in the claims hereinafter appended.

We claim:

1. A particle comprising:
at least one magnetic resonance imaging active material;
at least one fluorescent material; and
at least one polymer.

2. The particle of claim 1, wherein said at least one magnetic resonance imaging active material comprises a superparamagnetic material, a paramagnetic material, or combinations thereof.

3. The particle of claim 1, wherein said at least one magnetic resonance imaging active material comprises magnetite.

4. The particle of claim 3, wherein said iron oxide comprises magnetite crystals with a diameter of from about 1 to about 20 nm.

5. The particle of claim 1, wherein said particle comprises from about 40 to 65% magnetite by weight of the total particle.

6. The particle of claim 5, wherein said particle comprises about 63% magnetite by weight of the total particle.

7. The particle of claim 1, wherein said at least one fluorescent material comprises a fluorescein derivative.

8. The particle of claim 7, wherein said at least one fluorescent material comprises fluorescein isothiocyanate.

9. The particle of claim 1, wherein said particle comprises an amount of fluorescent material that results in a labelled cell having a mean fluorescence with an intensity two logs higher than an unlabeled cells

10. The particle of claim 1, wherein said at least one polymer comprises styrene, divinylbenzene, or combinations thereof.

11. The particle of claim 1, wherein said particle has a diameter of about 0.35 to about 2.5 μm.

12. The particle of claim 11, wherein said particle has a diameter of about 0.75 to about 1.25 μm.

13. The particle of claim 12, wherein said particle has a diameter of about 0.9 μm.

14. A method of tracking at least one cell in an environment comprising:
(a) labeling at least one cell with a particle according to claim 1;
(b) monitoring said at least one cell within said environment by monitoring said particle using at least one technique chosen from the group consisting of: magnetic resonance imaging (MRI), flow cytometry, fluorescent techniques, and microscopy.

15. The method of claim 14, wherein labeling at least one cell comprises uptake of said particle by said at least one cell.

16. The method of claim 15, wherein said uptake comprises phagocytosis, endocytosis or microinjection.

17. The method of claim 14, wherein said at least one cell is a hematopoietic cell, a lymphocyte, a mesenchymal stem cell, a muscle satellite cell, an embryonic stem cell, or a neural stem cell.

18. The method of claim 14, wherein said at least one magnetic resonance imaging active material comprises a superparamagnetic material, a paramagnetic material, or combinations thereof.

19. The method of claim 14, wherein said at least one magnetic resonance imaging active material comprises magnetite.

20. The method of claim 19, wherein said iron oxide comprises magnetite crystals with a diameter of from about 1 to about 20 nm.

21. The method of claim 14, wherein said particle comprises from about 40 to 65% magnetite by weight of the total particle.

22. The method of claim 21, wherein said particle comprises about 63% magnetite by weight of the total particle.

23. The method of claim 14, wherein said at least one fluorescent material comprises a fluorescein derivative.

24. The method of claim 23, wherein said at least one fluorescent material comprises fluorescein isothiocyanate.

25. The particle of claim 14, wherein said particle comprises an amount of fluorescent material that results in a labelled cell having a mean fluorescence with an intensity two logs higher than an unlabeled cells

26. The particle of claim 14, wherein said at least one polymer comprises styrene, divinylbenzene, or combinations thereof.

27. The method of claim 14, wherein said particle has a diameter of about 0.35 to about 2.5 μm.

28. The method of claim 27, wherein said particle has a diameter of about 0.75 to about 1.25 μm.

29. The method of claim 28, wherein said particle has a diameter of about 0.9 μm.
